(19) United States
(12) Patent Application Publication
(10) Pub. No.: US 2018/0099098 A1
(43) Pub. Date: Apr. 12, 2018

(54) DEVICE FOR INJECTING STEM CELLS

(71) Applicant: NEONEURON LLC, San Antonio, TX (US)

(72) Inventor: Marcel M. Daadi, Palo Alto, CA (US)

(73) Assignee: NEONEURON LLC, San Antonio, TX (US)

(21) Appl. No.: 15/727,583

(22) Filed: Oct. 7, 2017

Related U.S. Application Data
(60) Provisional application No. 62/405,897, filed on Oct. 8, 2016.

(51) Int. Cl. A61M 5/31; C12N 5/0735; A61K 35/545

(52) U.S. Cl. CPC A61M 5/3134; A61M 2005/3201; A61K 35/545; C12N 5/0606

(57) ABSTRACT

A multi-module or single-module cannula device for delivery of stems cells. The multi-module device has at least two cannulas of increasing diameter with larger cannulas encasing smaller cannulas. The single-module device has at least two cannula sections of increasing diameter. Both the multi-module and single-module devices have a first cannula (multi-module) or cannula section (single module) that has an injection end and an attachment end, both of which may be tapered or straight or one of each. The injection end is used for direct entry of the cannula device into a stem cell injection site and the attachment end is used to attach a medical attachment that is used to pump the cells through the device to the injection site. The increasing diameter of the cannulas or cannula sections of the device prevent backflow of the cells onto the exterior of the device. The device maybe used to deliver stem cells to a patient that is undergoing an MRI scan.

### CROSS-REFERENCE TO RELATED APPLICATIONS

This application claims priority to and the benefit under 35 U.S.C. 119(e) to U.S. Provisional Patent Application No. 62/405,897 filed on Oct. 8, 2016, the disclosure of which is incorporated by reference in its entirety herein.

### FIELD OF THE INVENTION

The present invention relates generally to cell therapy. More specifically, the present invention relates to devices and methods for injecting stem cells into the body of an organism under the guidance of a magnetic resonance imaging (MRI) scan.

### BACKGROUND OF THE INVENTION

Stem Cells are unspecialized cells within the body of an organism that have the potential to develop into any type of cell. When stem cells are sent to an area of injury or illness, they have the potential to rebuild and repair damaged tissue. Transplantation of stem cells into a patient requires that a medical practitioner obtain stem cells from the patient or from other human or non-human sources. The stem cells are isolated, purified, and administered to the area on the body of the patient that is being treated.

Optimal stem cell delivery procedures are critical to the success of the cell therapy approach. Variables such as flow rate, suspension solution, needle diameter, cell density, and tissue mechanics affect tissue penetration, backflow along the needle, and the dispersion and survival of injected cells during delivery. Most cell transplantation centers engaged in human clinical trials use custom-designed cannula needles, syringes, or catheters, sometimes barring the use of MRI-guided delivery to target tissue. As a result, stem cell therapies may be hampered because more than 80% of grafted cells do not survive the delivery to tissues, such as the heart, liver, pancreas, and brain, which translates to poor patient outcomes for patients needing therapy in these areas.

There is therefore, a current need in the art for cannulas that are compatible with MRI and optimal for injecting cells so that stem cells can be precisely deposited into the target areas a patient that is undergoing an MRI procedure.

### SUMMARY OF THE INVENTION

The present invention overcomes the need in the art by providing a device and method for injecting stem cells into an organism that is undergoing an MRI procedure.

In one embodiment, the present invention relates to a multi-module stem cell injection device comprising at least two cannulas with increasing diameters, the device comprising: (i) a first cannula comprising an injection end, an attachment end, an internal diameter in a range of about 100 μm to about 550 μm, and an outer diameter in a range of about 200 μm to about 700 μm; and (ii) a second cannula configured to encase the first cannula, wherein the second cannula comprises an injection end, an attachment end, an internal diameter in a range of about 300 μm to about 700 μm, and an outer diameter in a range of about 500 μm to about 900 μm, wherein the first cannula longer than the second cannula.

In another embodiment of the multi-module device, the first cannula is encased within the second cannula, the injection end of the first cannula extends about 1 mm to about 6 mm beyond the injection end of the second cannula, and the attachment end of the first cannula extends at least 1 cm beyond the attachment end of the second cannula.

In a further embodiment of the multi-module device, a third cannula is configured to encase the first and second cannula, wherein the third cannula has an injection end, an attachment end, an internal diameter in the range of about 700 μm to about 1000 μm, and an outer diameter in a range of about 800 μm to about 2000 μm, wherein the second cannula is longer than the third cannula.

In another embodiment of the multi-module device, the second cannula is encased within the third cannula and the injection end of the second cannula extends about 1 mm to about 20 mm beyond the injection end of the third cannula.

In a further embodiment of the multi-module device, the device further comprises one or more additional cannulas each having an injection end and an attachment end and configured to encase a smaller cannula of the device.

In another embodiment of the multi-module device, the injection end and the attachment end of the first cannula are both straight, both tapered, or one of the injection end or the attachment end is straight and the other end is tapered.

In a further embodiment of the multi-module device, the injection end of the first cannula is configured to deliver a biological material directly into a source and the attachment end of the first cannula and/or the first and second cannulas are configured to accept a medical attachment that will push a biological material through the device to the injection end of the first cannula for direct delivery to the source.

In another embodiment, the present invention relates to a single-module stem cell injection device comprising a single cannula with at least two sections, wherein each section has an increasing diameter, the device comprising: (i) a first section comprising an injection end, an attachment end, and an inner diameter in the range of about 100 μm to about 550 μm; and (ii) a second section comprising an injection end, an attachment end, and an outer diameter in the range of about 300 μm to about 700 μm, wherein the first section is longer than the second section.

In one embodiment of the single-module device, the injection end of the first section extends about 1 mm to about 6 mm beyond the injection end of the second section and the attachment end of the first section extends at least 1 cm beyond the attachment end of the second section.

In another embodiment of the single-module device, the device further comprises a third section comprising an injection end, an attachment end, and an outer diameter in the range of about 800 μm an to about 2000 μm.

In a further embodiment of the single-module device, the device further comprises one or more additional sections each having an injection end and an attachment end.

In another embodiment of the single module device, the injection end and the attachment end of the first section are both straight, both tapered, or one of the injection end or the attachment end is straight and the other end is tapered.

In a further embodiment of the single-module device, the injection end of the first section is configured to deliver a biological material directly into a source and the attachment end of the first section and/or the first and second sections are configured to accept a medical attachment that will push a biological material through the device into the injection end of the first section for direct delivery to the source.

In another embodiment, the multi-module or the single-module device is about 6 cm to about 30 cm in length.

In a further embodiment, the multi-module or single-module device is made of an MRI-compatible material selected from the group consisting of polymer, silicon, fused silica, fiber optic, plastic, glass, and ceramic.

In another embodiment, the present invention relates to a method comprising administering stem cells into an organism with the multi-module stem cell injection device, wherein the first cannula is encased within the second cannula, the method comprising the steps of: (i) obtaining stem cells from an organism; (ii) loading the stem cells into the first cannula of the device; (iii) attaching a medical attachment to the attachment end of the first cannula of the device; (iv) administering the injection end of the first cannula to the same or different organism, wherein the medical attachment is used to push the stem cells through the injection end of the first cannula into the same or different organism, wherein the increasing diameters of the at least two cannulas of the device prevent backflow of the stem cells onto the exterior of the device.

In a further embodiment, the present invention relates to a method comprising administering stem cells into an organism with the single-module stem cell injection device comprising a single cannula with at least two sections, wherein the method comprises the steps of: (i) obtaining stem cells from an organism; (ii) loading the stem cells into the first section of the device; (iii) attaching a medical attachment to the attachment end of the first section of the device; (iv) administering the injection end of the first section to the same or different organism, wherein the medical attachment is used to push the stem cells through the first section into the same or different organism, wherein the increasing diameters of the at least two sections of the device prevent backflow of the stem cells onto the exterior of the device.

Additional aspects and embodiments of the invention will be provided, without limitation, in the detailed description of the invention that is set forth below.

### BRIEF DESCRIPTION OF THE DRAWINGS

FIG. 1A shows a multi-module cannula device with three individual cannulas that are configured to be stacked to form a single stem cell delivery device.

FIG. 1B shows the multi-module cannula device of FIG. 1A where the three individual cannulas have been non-permanently or permanently attached with an adhesive.

FIG. 2 shows a single-module cannula device with three different length sections that is produced as a single piece.

FIG. 3 shows a multi-module cannula device with three individual cannulas where the innermost cannula has tapered injection and attachment ends.

FIG. 4 shows a single-module cannula device with three different length sections that is produced as a single piece where the innermost module has tapered injection and attachment ends.

### DETAILED DESCRIPTION OF THE INVENTION

Set forth below is a description of what are currently believed to be preferred embodiments of the claimed invention. As used in this specification and the appended claims, the singular forms “a,” “an,” and “the” include plural referents unless the context clearly dictates otherwise.

As used herein, the term “cannula” refers to a thin tube with two open ends that is intended to be inserted into the tissue of a patient in order to retrieve and/or administer stem cells.

As used herein, the term “about” is meant to be used in its traditional sense to mean “approximately”; thus, where a measure, such as “about 1 cm” is used that measure is meant to include small deviations on either side of that measurement where such deviations would be obvious deviations to those of ordinary skill in the art to which the invention pertains.

As used herein, the term “patient” is meant to refer to any live organism that is subject to a procedure where stem cells are being injected into the live organism or a target area of the live organism. Patients contemplated within the present invention include any human or animal species, including non-human mammals as well as any vertebrate or invertebrate species. Where appropriate, the term “organism” or “source” may be used interchangeably with the term “patient.”

The device of the present invention is directed to a multi-module or single-module cannula for injecting stem cells directly into live or dead tissue of a patient. A particular advantage of the device is that the stem cells may be injected into a patient while the patient is undergoing an MRI scan. Examples 1 and 2 describe how the device of the present invention may be used to administer stem cells to the brain (Example 1) and heart (Example 2) of a patient while under the guidance of an MRI scan.

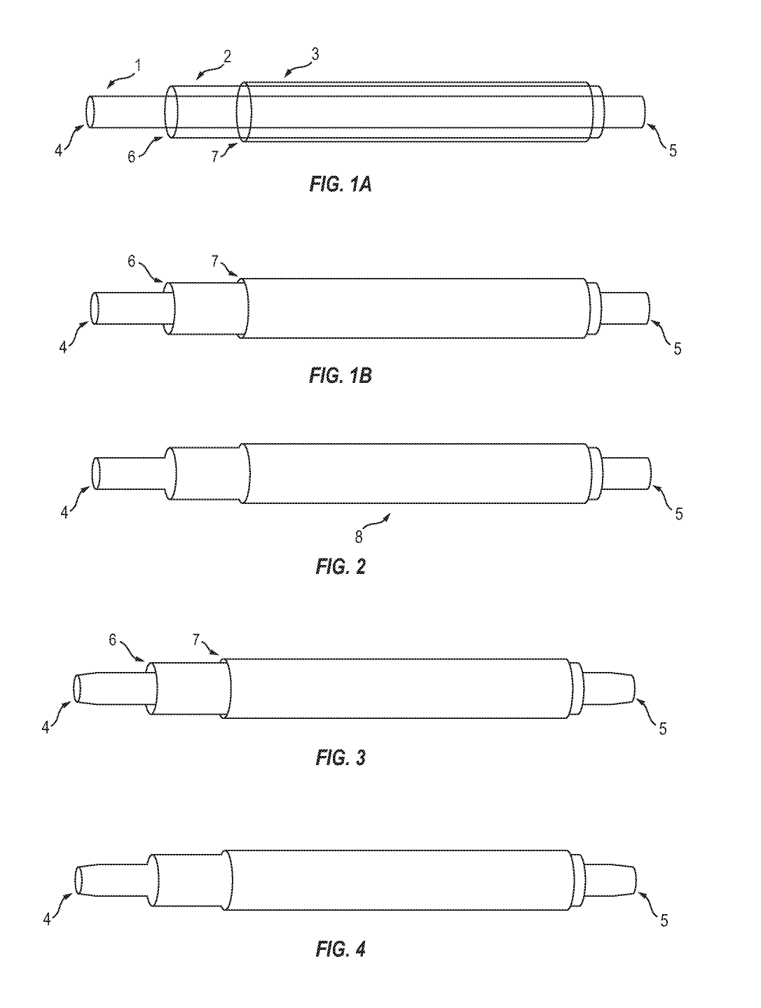

In one embodiment, the device is a multi-module cannula comprised of at least two cannulas of increasing diameter wherein the cannula with the larger diameter encloses the cannula with the smaller diameter (FIGS. 1A, 3). The multi-module device may comprise (i) a first cannula comprising an injection end, an attachment end, an internal diameter in a range of about 100 μm to about 550 μm, and an outer diameter in a range of about 200 μm to about 700 μm; and (ii) a second cannula configured to encase the first cannula, wherein the second cannula comprises an injection end, an attachment end, an internal diameter in a range of about 300 μm to about 700 μm, and an outer diameter in a range of about 500 μm to about 900 μm, wherein the first cannula is longer than the second cannula.

In a further embodiment, the multi-module cannula device includes a third cannula configured to encase the first and second cannula, wherein the third cannula has an injection end, an attachment end, an internal diameter in the range of about 700 μm to about 1000 μm, and an outer diameter in a range of about 800 μm to about 2000 μm, wherein the second cannula is longer than the third cannula. The multi-module cannula device may comprise one or more additional cannulas each having an injection end and an attachment end and configured to encase a smaller cannula of the device. Where the second cannula is encased within the third cannula, the injection end of the second cannula may extend about 1 mm to about 20 mm beyond the injection end of the third cannula.

In the multi-module cannula devices, the individual cannulas are attached to each other using an adhesive, such as for example, medical grade epoxy.

In another embodiment, the device is a single-module cannula device comprised of at least two cannula sections, wherein each of the cannula sections has an increasing diameter, the device comprising: (i) a first cannula section comprising an injection end, an attachment end, and an inner diameter in the range of about 100 μm to about 550 μm; and (ii) a second larger cannula section fused to the first cannula section comprising an injection end, an attachment end, and an outer diameter in the range of about 300 μm to about 700 μm, wherein the inner cannula section is longer than the outer cannula section. As is shown in FIGS. 1B and 4, the individual cannula sections of the single-module device have increasing diameters in a stepped or graded configuration.

In another embodiment, the single-module cannula device further comprises a third cannula section fused to the second cannula section comprising an injection end, an attachment end, and an outer diameter in the range of about 800 μm to about 2000 μm. The single-module cannula device may comprise one or more additional cannula sections each having an injection end and an attachment end and configured to be fused to a smaller cannula section of the device.

In a further embodiment, the injection end of the first cannula (multi-module) or cannula section (single-module) of the device, respectively, is configured to deliver a biological material directly into a source. In another embodiment, the attachment end of the device is configured to accept a medical attachment that will push a biological material through the device to the injection end of the device for direct delivery to a source. The biological material may be a cell suspension, such as a stem cell suspension, a cluster of cells, or tissue pieces. The source may be any organism. The medical attachment, which may be cell infusion tubing, a catheter, a syringe, or a microsyringe, may be attached to the attachment end either directly or via a luer lock adapter.

In the multi-module device, the medical attachment may attach to the first cannula or the first and second cannulas. In the single-module device, the medical attachment may attach to the first cannula section or the first and second cannula section.

In either the multi-module or single-module cannula devices, the injection end and the attachment end of the first cannula or first cannula section may be straight, tapered, or have one tapered and one straight end. The choice to use a tapered or straight end on the injection end will depend on the tissue that is being administered the stem cells. Tissue that has high porosity or delicate mechanical properties will be more receptive to tapered ends since they will more readily penetrate the tissue, thus reducing any potential trauma to the tissue. The choice to use a tapered or straight end for the attachment end of the first and/or first and second cannula(s) or cannula section(s) may depend on the attachment that will be used to push the stem cells through the internal cannula to the tissue site. In this regard, it may be preferable in some circumstances to have a device comprising a first cannula or cannula section that has a tapered injection end and a straight attachment end.

In either the multi-module or single-module cannula devices, the injection end of the first cannula (multi-module) or first cannula section (single-module) may extend about 1 mm to about 6 mm beyond the injection end of the second cannula or cannula section, respectively. The distance of the injection end of the first cannula or cannula section to the second cannula or cannula section will be variable depending upon the target tissue where the cells will be injected. For target tissues in small organs, such as the brain of a small animal or human child, the length of the injection tip may need to be very small at just 1-2 mm, whereas for a larger organ, such as a human or large mammal brain or heart, the injection tip may be larger at 5-6 mm.

In the multi-module or single-module cannula devices, the attachment end of the first cannula or cannula section may extend at least 1 cm beyond the attachment end of the second cannula. The distance between the second cannula or cannula section and the third cannula or cannula section can have any variation, ranging from a flush end for the two sections versus a second cannula or cannula section that extends 1 or 2 cm or more beyond the third cannula or cannula section. The distance between the second and third cannulas or cannula sections will depend on the dimensions and shape of the medical attachment device.

The overall length of the multi-module or single-module cannula devices described herein will also vary based upon the size of the organism and indication for which the device is being used. Reasonable lengths for the entire device, including all cannula sections, may range from about 6 cm to about 30 cm in length.

Where the device is to be used in conjunction with an MRI-scan, the device may be made of an MRI-compatible material selected from the group consisting of polymer, silicon, fused silica, fiber optic, plastic, glass, and ceramic. The material may be arranged vertically, horizontally, circularly, or in angular fashion in a mesh pattern or by a combination of these arrangements. In one embodiment, the single-module cannula device may be manufactured or 3-D printed as a single piece.

The device of the present invention has utility in many applications. For example, the device may be used to inject stem cells into live or dead tissue or a body part of a patient in order to repair injured or damaged tissue or to repair a body part that is subject to disease and/or injury. Examples of live or dead tissue or body parts that can be used with the device of the present invention include without limitation, the brain or brain tissue, the heart or cardiac tissue, muscles, skin, blood vessels, the pancreas, the liver, kidneys, eyes, ears, and articulations such as the knees. A particularly useful application of the device of the of the present invention is for the direct targeting and delivery of stem cells to a tissue or body part of a patient while the patient is undergoing an MRI scan.

In another embodiment, administration of stem cells to a patient undergoing MRI with the multi-module or single-module cannula device of the present invention includes the steps of: (i) obtaining stem cells from an organism; (ii) loading the stem cells into the injection end of the device; (iii) attaching a medical attachment to the attachment end of the device, wherein upon activation, the medical attachment pushes the stem cells through the device to the injection end; (iv) inserting the injection end of the device directly into a tissue or organ of the same or different organism; and (v) administering the stem cells to the same or different organism by activating the medical attachment, wherein the increasing diameters of the at least two cannula sections of the device prevent the stem cells from backing up onto the exterior of the device.

Stem cells for use with the device of the present invention may be obtained from any organism living or dead. To prepare the stem cells, biopsies are taken from a stem cell source of the organism. Examples of stem-cells sources include without limitation, blood, bone marrow, dental tissue, skin tissue, adipose tissue, placenta, cord blood, pluripotent stem cells, cardiomyocytes, and neural or brain tissue. Once obtained from the source, the stem cells are differentiated for up to weeks or months prior to use on a patient. Where the stems cells are obtained from a species that is different from the target species, the stem cells may require immunosuppression. For example, stem cells obtained from a pig for ultimate administration to a human will require immunosuppression from the time the cells are obtained through administration and continuing thereafter. Full immunosuppression may also be required for allogeneic cells obtained from one unrelated member of a species to another member of the same species. By contrast, where stem cells are obtained from a patient for readministration to that same patient, immunosuppression may only be required for a limited time, such as for example, six months to a year following the stem cell transplant. Where the stem cells are to be used on brain tissue, the degree of immunosuppression may be less than that required for other organs because brain tissue is known to be immunoprivileged due to the blood-brain barrier; thus, once the stem cells are injected into the brain, they will generally not trigger an immune response. Once the stem cells are administered to a site of damaged or injured tissue, the stem cells attach to the site and begin generation of new tissue.

It is to be understood that while the invention has been described in conjunction with the embodiments set forth above, the foregoing description is intended to illustrate and not limit the scope of the invention. Further, it is to be understood that the embodiments set forth herein are not exhaustive and that modifications and variations of the invention will be apparent to those of ordinary skill in the art without departing from the scope and spirit of the invention.

### EXPERIMENTAL

The following examples are set forth to provide those of ordinary skill in the art with a complete disclosure of how to make and use the aspects and embodiments of the invention as set forth herein.

### EXAMPLE 1

### Administration of Neural Stem Cells Into the Brain of a Human Patient

Stem cells obtained from the brain of a dead pig are differentiated into dopamine neurons and treated with immunosuppressants. The stem cells are to be injected into the brain of a human patient with a neurological disorder or injury.

The patient with the neurological disorder or injury is prepared for an MRI by being placed into the MRI machine in a supine position. The MRI is equipped with MRI mapping software, which will allow the clinicians to identify the precise location of the patient's brain injury.

Prior to the initiation of the MRI procedure, the stem cells are prepared for administration by being moved from the laboratory where they are kept to the surgical facility. In the surgical facility, a multi-module cannula having three separate cannulas with the first internal cannula having a 100 μm inner diameter and a 200 μm outer diameter, the second intermediate cannula having a 300 μm inner diameter and a 500 μm outer diameter, and a third outer cannula having a 600 μm inner diameter and a 800 μm outer diameter. The attachment end of the multi-module cannula is attached to an injection syringe, either directly or through a tubing, which in turn is attached to a cell pump, the latter of which will be used to both fill the cannula device and set the amount and rate of the stem cells that will be delivered to the targeted brain tissue once the procedure begins. For the brain tissue procedure, 1 mL of the stem cell suspension is uploaded into the first cannula of the multi-module cannula device. Depending on the brain tissue disorder or injury, an amount less than 1 mL of cell suspension may be preferable to a full 1 mL. To fill the cannula device, the injection tip of the cannula is inserted into the stem cell suspension and the cell pump is set at an aspiration rate of 10 μL/minute. If appropriate for the aspiration of the cell suspension and/or the timing of the procedure, a rate anywhere from 0.1-100 μL/minute may be used. Once the cannula has aspirated the required volume of cell suspension, the device is ready for administration to the patient.

The MRI procedure is initiated on the patient and the exact coordinates of the patient's brain injury is identified by the MRI mapping software. The surgeon who is administering the stem cells proceeds to gain access to the patient's brain by incising the skin on the patient's scalp and drilling a bore towards the precise area of the brain injury and placing a guiding device on the patient's head that will facilitate the insertion of the cannula into the target area of the patient's brain. Once the patient's position is certain, the cell pump is turned on and using a guiding device, the surgeon inserts the injection end of the cannula directly into the patient's brain toward the target coordinates. During the procedure, an MRI scan can be performed in order to ensure that the cannula is being placed within the target coordinates of the patient's brain. The cell pump is turned on at a rate of 10 μL/minute and set to deliver the target volume of 1 mL of stem cells to the patient's brain. Depending on the circumstances of the procedure and/or the health of the patient, a different rate of cell delivery, such as a rate ranging from 0.1 μL-100 μL/minute. Once the target volume of the stem cell suspension has been transplanted to the patient's brain, the pump is turned off, the procedure is completed, and the patient is removed from the MRI machine and treated post-operatively as appropriate.

### EXAMPLE 2

### Administration of Cardiac Stem Cells Into a Ventricle of the Heart of a Human Patient

Stem cells obtained from bone marrow of a dead pig are differentiated into cardiac cells and treated with immunosuppressants. The stem cells are to be injected into the ventricle wall of the heart of a human patient that has had a cardiac injury.

The patient with the cardiac injury is prepared for an MRI by being placed into the MRI machine in a supine position. The MRI is equipped with MRI mapping software, which will allow the clinicians to identify the precise location of the patient's cardiac injury.

Prior to the initiation of the MRI procedure, the stem cells are prepared for administration by being moved from the laboratory where they are kept to the surgical facility. In the surgical facility, a multi-module cannula having three separate cannulas with the first internal cannula having a 400 μm inner diameter and a 600 μm outer diameter, the second intermediate cannula having a 700 μm inner diameter and a 1000 μm outer diameter, and a third outer cannula having a 1100 μm an inner diameter and a 1500 μm an outer diameter. The attachment end of the multi-module cannula is attached to an injection syringe, which in turn is attached to a cell pump, the latter of which will be used to both fill the cannula device and set the amount and rate of the stem cells that will be delivered to the targeted cardiac tissue once the procedure begins. For the cardiac procedure, 10 mL of stem cell suspension will be used. Depending on the cardiac injury and the type of injection, i.e., directly into the ventricular wall versus systemic administration into the ventricle, anywhere from 0.1 mL to 100 mL of stem cell suspension may be used. To fill the cannula device, the injection tip of the first cannula section is inserted into the stem cell suspension and the cell pump is set at an aspiration rate of 50 μL/minute. If appropriate for the aspiration of the cell suspension and/or the timing of the procedure, a rate anywhere from 0.1-100 μL/minute may be used. Once the cannula has aspirated the target volume of the cell suspension, the device is ready for administration to the patient.

The MRI procedure is initiated on the patient and the exact coordinates of the patient's cardiac injury is identified by the MRI mapping software. The surgeon who is administering the stem cells proceeds to gain access to the patient's heart by incising the skin on the patient's chest and if needed, drilling a bore towards the precise area of the cardiac injury. A guiding device is placed on the patient's chest to facilitate the introduction of the cannula into the target area within the patient's heart. Once the position of the injury is certain, the cell pump is turned on and using the guiding device, the surgeon inserts the injection end of the cannula directly into the ventricular wall toward the target coordinates of the damaged tissue. During the procedure, an MRI scan can be performed in order to ensure that the cannula is being placed within the target coordinates of the ventricular wall. The cell pump is turned on at a rate of 100 μL/minute and set to deliver the 10 mL of stem cells to the patient's heart. Depending on the circumstances of the procedure and/or the health of the patient, a different rate of cell delivery, such as a rate ranging from 0.1 μL-1 mL/minute may be preferable. Once the target volume of stem cells have been transplanted to the patient's heart, the pump is turned off, the procedure is completed, and the patient is removed from the MRI machine and treated post-operatively as appropriate.

I claim:

1. A multi-module cannula device comprising at least two cannulas with increasing diameters, the device comprising:
(i) a first cannula comprising an injection end, an attachment end, an internal diameter in a range of about 100 μm to about 550 μm, and an outer diameter in a range of about 200 μm to about 700 μm; and
(ii) a second cannula configured to encase the first cannula, wherein the second cannula comprises an injection end, an attachment end, an internal diameter in a range of about 300 μm to about 700 μm, and an outer diameter in a range of about 500 μm to about 900 μm, wherein the first cannula is longer than the second cannula.

2. The device of claim 1, wherein the first cannula is encased within the second cannula, the injection end of the first cannula extends about 1 mm to about 6 mm beyond the injection end of the second cannula, and the attachment end of the first cannula extends at least 1 cm beyond the attachment end of the second cannula.

3. The device of claim 1, wherein the injection end and the attachment end of the first cannula are both straight, both tapered, or one of the first or attachment end is straight and the other end is tapered.

4. The device of claim 1, wherein the injection end of the first cannula is configured to deliver a biological material directly into a source and the attachment end of the first cannula and/or the first and second cannulas are configured to accept a medical attachment that will push a biological material through the first cannula to the injection end of the first cannula for direct delivery to the source.

5. The device of claim 4, wherein the medical attachment is selected from the group consisting of cell infusion tubing, a catheter, a syringe, and a microsyringe.

6. The device of claim 1, further comprising:
(iii) a third cannula configured to encase the first and second cannula, wherein the third cannula has an injection end, an attachment end, an internal diameter in the range of about 700 μm to about 1000 μm, and an outer diameter in a range of about 800 μm to about 2000 μm, wherein the second cannula is longer than the third cannula.

7. The device of claim 6, wherein the second cannula is encased within the third cannula and the injection end of the second cannula extends about 1 mm to about 20 mm beyond the injection end of the third cannula.

8. The device of claim 8, further comprising one or more additional cannulas each having an injection end and an attachment end and configured to encase a smaller cannula of the device.

9. The device of claim 1, wherein the device is about 6 cm to about 30 cm in length.

10. The device of claim 1, wherein the device is made of an MRI-compatible material selected from the group consisting of polymer, silicon, fused silica, fiber optic, plastic, glass, and ceramic.

11. A single-module cannula device comprising a single cannula with at least two sections, wherein each of the sections has an increasing diameter, the device comprising:
(i) a first section comprising an injection end, an attachment end, and an inner diameter in the range of about 100 μm to about 550 μm; and
(ii) a second section comprising an injection end, an attachment end, and an outer diameter in the range of about 300 μm to about 700 μm,
wherein the first cannula section is longer than the second cannula section.

12. The device of claim 11, wherein the injection end of the first section extends about 1 mm to about 6 mm beyond the injection end of the second section and the attachment end of the first section extends at least 1 cm beyond the attachment end of the second section.

13. The device of claim 11, wherein the injection end and the attachment end of the first section are both straight, both tapered, or one of the first or attachment end is straight and the other end is tapered.

14. The device of claim 11, wherein the injection end of the first section is configured to deliver a biological material directly into a source and the attachment end of the first section and/or the first and second sections are configured to accept a medical attachment that will push a biological material through the device to the injection end of the first section for direct delivery to the source.

15. The device of claim 11, further comprising:
(iii) a third section comprising an injection end, an attachment end, and an outer diameter in the range of about 800 μm to about 2000 μm.

16. The device of claim 15, further comprising one or more additional sections each having an injection end and an attachment end.

17. The device of claim 11, wherein the device is about 6 cm to about 30 cm in length.

18. The device of claim 1, wherein the device is made of an MRI-compatible material selected from the group consisting of polymer, silicon, fused silica, fiber optic, plastic, glass, and ceramic.

19. A method comprising administering stem cells into an organism with the multi-module cannula device of claim 1, wherein the first cannula is encased within the second cannula, the method comprising the steps of:
(i) obtaining stem cells from an organism;
(ii) loading the stem cells into the injection end of the first cannula of the device;
(iii) attaching a medical attachment to the attachment end of the first cannula and/or the first and second cannula of the device;
(iv) administering the injection end of the first cannula to the same or different organism, wherein the medical attachment is used to push the stem cells through the device and into the same or different organism, wherein the increasing diameters of the at least two cannulas of the device prevent backflow of the stem cells onto the exterior of the device.

20. A method comprising administering stem cells into an organism with the single-cannula device of claim 11, wherein the method comprises the steps of:
(i) obtaining stem cells from an organism;
(ii) loading the stem cells into the injection end of the first section of the device;
(iii) attaching a medical attachment to the attachment end of the first section and/or the first and second section of the device;
(iv) administering the injection end of the first section to the same or different organism, wherein the medical attachment is used to push the stem cells through the device and into the same or different organism, wherein the increasing diameters of the at least two sections of the device prevent backflow of the stem cells onto the exterior of the device.
